(19) United States
(12) Patent Application Publication
(10) Pub. No.: US 2019/0321656 A1
(43) Pub. Date: Oct. 24, 2019

(54) Rectal Balloon with Sensor Cable

(71) Applicant: AngioDynamics, Inc., Latham, NY (US)

(72) Inventors: John ISHAM, Houston, TX (US); Tamsen VALOIR, Houston, TX (US)

(73) Assignee: AngioDynamics, Inc., Latham, NY (US)

(21) Appl. No.: 16/445,947

(22) Filed: Jun. 19, 2019

Related U.S. Application Data
(62) Division of application No. 14/065,127, filed on Oct. 28, 2013.
Application No. 14/065,127 is a continuation-in-part of application No. 13/591,546, filed on Aug. 22, 2012, now Pat. No. 9,126,035.
Application No. 13/591,546 is a continuation-in-part of application No. 13/444,626, filed on Apr. 11, 2012, now Pat. No. 8,603,129.
Application No. 13/444,626 is a continuation-in-part of application No. 12/141,270, filed on Jun. 18, 2008, now abandoned.
Application No. 12/141,270 is a continuation-in-part of application No. 12/034,470, filed on Feb. 20, 2008, now Pat. No. 8,080,031.
Application No. 12/034,470 is a continuation-in-part of application No. 11/966,544, filed on Dec. 28, 2007, now abandoned.
Application No. 11/966,544 is a continuation-in-part of application No. 11/933,018, filed on Oct. 31, 2007, now abandoned.
Application No. 11/933,018 is a continuation-in-part of application No. 11/623,702, filed on Jan. 16, 2007, now abandoned.
Application No. 14/065,127 is a continuation-in-part of application No. 13/299,348, filed on Nov. 17, 2011, now Pat. No. 8,679,147.

(51) Int. Cl. A61N 5/10; A61B 5/11

(52) U.S. Cl. CPC A61N 5/1031; A61B 5/1126; A61B 2017/22069; A61N 5/1071; A61N 5/10

(57) ABSTRACT

An endorectal balloon having a pocket thereon for holding a sensor cable that can be used for radiation dosimetry or to detect motion of the prostate or balloon.

### PRIOR RELATED APPLICATIONS

The present application is a continuation-in-part (CIP) of U.S. Ser. No. 13/444,626, filed on Apr. 11, 2012, allowed, which is a CIP of U.S. Ser. No. 12/141,270, filed on Jun. 18, 2008, abandoned, which is a CIP of U.S. Ser. No. 12/034,470, filed on Feb. 20, 2008, now patented as U.S. Pat. No. 8,080,031, which is CIP of U.S. Ser. No. 11/933,018, filed on Oct. 31, 2007, abandoned, which is a CIP of U.S. Ser. No. 11/623,702, filed on Jan. 16, 2007, abandoned, and all of which are incorporated by reference herein in their entirety for all purposes.

The present application is also a CIP of Ser. No. 13/299,348, filed Nov. 17, 2011, pending, which is a CIP of U.S. application Ser. No. 12/707,389, filed Feb. 17, 2010, now issued as U.S. Pat. No. 8,500,771, which is a CIP of U.S. application Ser. No. 12/412,017, filed Mar. 26, 2009, abandoned, which is a CIP of U.S. application Ser. No. 12/410,639 filed on Mar. 25, 2009, now issued as U.S. Pat. No. 8,454,648 on Jun. 4, 2013, which is a CIP of U.S. application Ser. No. 12/141,270 filed on Jun. 18, 2008, abandoned, which is a CIP of U.S. application Ser. No. 12/034,470, filed Feb. 20, 2008, now issued as U.S. Pat. No. 8,080,031, which is a CIP of U.S. application Ser. No. 11/966,544 filed on Dec. 28, 2007, abandoned, which is CIP of U.S. Ser. No. 11/933,018, filed on Oct. 31, 2007, abandoned, which is a CIP of U.S. Ser. No. 11/623,702, filed on Jan. 16, 2007, abandoned, and all of which are incorporated by reference herein in their entirety for all purposes.

The is invention is a CIP of Ser. No. 13/591,546, filed Aug. 22, 2012, pending, which is also incorporated by reference herein in its entirety.

### FIELD OF THE INVENTION

The present invention relates to endorectal balloons that are used for immobilizing the region surrounding the prostate during pre-treatment simulation and target localization, as well as during the delivery of radiation therapy to treat prostate cancer. More particularly, the present invention relates to balloon specially designed to have a pocket for holding data cable therein. The data cable can be for any types of sensor, and preferably is a non-implantable electromagnetic rectal sensors that can accurately monitor the movement during a radiation therapy, or a non-implantable plastic scintillator dosage sensor that can monitor dosimetry during therapy.

### BACKGROUND OF THE INVENTION

Treatment of prostate cancer using radiation therapy is difficult due to the prostate's position near radiation-sensitive tissues and is further complicated by surprising levels of prostate motion.

During external beam radiation therapy (XRT), radiation is directed along different axes to the target prostate, which is near the rectal wall. Where the beams cross, the radiation dose is the highest, and thus the prostate can be preferentially targeted. Misdirected radiation beams may perforate the rectal wall causing radiation proctitus (rectal bleeding), as well as erectile dysfunction (ED), incontinence and other complications. In fact, as many as half the treated men suffer from ED and/or incontinence.

A major factor limiting radiation oncologists' attempts to reduce the volume of the anterior rectal wall and other healthy tissues receiving a high radiation dose is the position of the prostate gland as well as the intrinsic motion up to 10 mm in the anterior to posterior direction caused by rectal peristalsis. Accordingly, oncologists generally will add a margin to the radiation field in order to ensure that the entire prostate gland receives the prescription dose. This margin is typically on the order of 5 to 15 mm. As a consequence, lower doses of radiation may need to be used so as not to overexpose healthy structures. However, this may lead to inadequate radiation treatment and a higher probability of local cancer recurrence.

US20030028097 by MedRad describes an rectal balloon to help immobilize the prostate during treatment. One of the problems with the MedRad design is the discomfort associated with installing the rectal balloon within the rectal cavity. In particular, a relatively sturdy and wide diameter shaft is connected to a relatively large thick-walled balloon. Because the balloon is not supported by anything other than by the shaft, the balloon is formed of a relatively rugged and thick material. The resulting relatively large size and stiffness of the balloon causes considerable discomfort for the patient.

A second, and more important, problem with the MedRad rectal balloon is that it is “non-conforming.” Thus, when squeezed, the shape of the balloon is lost, because there are no interior welds restraining the balloon. Thus, even if shaped when lightly inflated, the shape is lost when squeezed or when placed in the constrained environment of the rectum. Thus, the prostate can easily slide off its surface, and the balloon does not sufficiently immobilize the prostate.

Because of these problems, a need arose for a rectal balloon that retains the prostate in a fixed position when the balloon is in a fully inflated and/or squeezed or constrained condition. A balloon that can retain a shape, even when squeezed or otherwise constrained, is known as a “conforming” balloon.

U.S. Pat. No. 8,080,031 and related applications describe a rectal balloon that is conforming. This balloon has an interior weld that restrains the balloon such that it does not lose shape, even when squeezed in the highly mobile environment of the rectum. In more detail, the balloon is made of three layers, wherein the middle layer is connected to the top layer to provide a central groove which provides the dimpled or grooved seating area into which the prostate is wedged. The weld is shifted distally slightly, so that there is a bit more material proximal to the weld, which when overinflated stretches more, providing a proximal bulge, serving to further wedge the seminal vesicles into place.

However, there are many other ways of making a conforming balloon, and US201301009906, incorporated by reference herein, discusses a few additional such ways. For example, the restrained layer of the balloon can be welded to the central shaft or lumen, instead of a middle layer, and this would also provide a central seating area for the prostate and a conforming shape under constraint. Likewise, the surface can be pinched and welded to itself, or to a baffle, and combinations are also possible.

As discussed above, another important consideration when treating patients using radiation therapy is that the proper dose of radiation reaches the treatment site. This is very important whether the treatment method utilizes implanted radiation seeds, brachytherapy, external beams of radiation, proton particle delivery or any other form of high energy treatment. Excessive dosing of the patient can lead to severe side effects including impotence and urinary incontinence. Thus, a proper treatment plan should deliver an adequate amount of radiation to the treatment site while minimizing the dose delivered to the surrounding tissues, and it would be advantageous to the medical practitioner to know the actual dosage being delivered. and/or the position of the internal organs during radiation delivery.

U.S. Pat. No. 6,963,771 describes an implantable device for radiation dose verification. The method includes (a) placing at least one wireless implantable sensor in a first subject at a target location; (b) administering a first dose of radiation therapy into the first subject; (c) obtaining radiation data from the at least one wireless implantable sensor; and (d) calculating a radiation dose amount received by the first subject at the target location based on the radiation data obtained from the at least one wireless sensor during and/or after exposure to the first administered dose of radiation to determine and/or verify a dose amount of radiation delivered to the target location. However, the use of implantable medical devices is not an optimum solution.

U.S. Pat. No. 7,361,134 teaches a method of determining the dose by locating three or more detectors in the vicinity of a seed source of radiation. Each of the detectors provides an output indicative of the amount of radiation received from the source and complex calculations determine the location of the source from the detector outputs. However, this detector system is for brachytherapy and the detector is applied to tissue via a needle (or multiple needles), not a prostate immobilizing balloon. Further, the system cannot detect radiation in real time, and the sensor is not water equivalent.

U.S. Pat. Nos. 7,662,083 and 8,133,167 teach another sensor for brachytherapy that uses plastic scintillators coupled to optical fibers in the sensor portion. The patent does contemplate using a balloon for delivering the sensor, but no details are provided. The balloon 610 shown appears to lack any structure and be non-confirming, and therefore, would not suffice to immobilize the prostate. Additionally, the sensors and accompanying catheters need to be implanted inside a patient's body, which greatly increases the discomfort and inconvenience in practical application.

US20120068075 by Beddar provides an apparatus and methods for measuring radiation levels in vivo in real time, including a scintillating material coupled to a retention member, which could be a catheter or balloon. However, this system is highly simplistic and cannot immobilize the prostate during therapy. Indeed, the balloon 91 shown appears the same as the MedRad balloon, and can be expected to have similar shortcomings.

U.S. Pat. No. 8,183,534, also by Beddar, teaches an array of dosimeters, similar to those above, wherein the array allows a unique calibration method to be employed, as well as allowing assessment of complex, two-dimensional field patterns, such as might be encountered in IMRT and tomotherapy. However, the complex array of sensors contributes to complexity, cost and size of the device, none of which are desirable.

Therefore, there is the need for a rectal balloon that can both immobilize the prostate and be equipped with a properly positioned radiation sensor and/or motion sensor, such that the radiation dose and movement can both be monitored during treatment.

### SUMMARY OF THE DISCLOSURE

The disclosure provides an endorectal balloon that immobilizes the prostate for e.g., external beam radiation therapy, and also has pockets thereon or therein for holding a cable sensor, such as a motion and/or radiation sensor.

The balloon generally comprises a shaft having a fluid passageway extending at least partway therethrough. A balloon is affixed over an end of the shaft such that the fluid passageway communicates with an interior of the balloon. The balloon also is conforming and has a conforming depression on a top surface thereof, while the bottom surface is generally rounded to push the opposite rectal wall away from the target treatment area.

The conforming depression is made with an interior weld, and the weld can be to a middle layer, to the lumen, to itself, to a baffle, or combinations thereof. The conforming depression can be in the shape of a groove or a dimple, although a groove is currently preferred.

Co-located with the conforming depression or groove, is a pocket or channel into which a sensor can be fit. The pocket can be formed as part of the top layer weld (e.g., a U-shaped weld will form a pocket) to a middle layer or lumen, or another layer or strip can be added to make a pocket. The sensor and cable either runs through the lumen to the pocket, or can run outside the lumen to the pocket. Alternatively, or in addition, a pair of pockets can be placed on either side of the groove.

Where the sensor cable runs alongside the lumen or shaft (instead of inside it) an attachment means is also provided, e.g., a reversible locking clip or snap fit clip. This allow the sensor to be affixed to the shaft and holds the sensor in place during insertion into the rectum, yet the sensor can be removed after use and saved for the next procedure, while the balloon is disposed of.

The balloon is of course fitted with means for introducing air or other fluid such as water or contrast, and keeping the fluid therein, and these can be of any shape or design known in the art. Typical means for introducing fluids is a lumen or flexible tube with stop cock or other valve means and connector for fluidly connecting to a syringe or other air or fluid source. Alternatively, a luer lock can be used in place of stock cock and luer connector.

For rectal purposes the balloon is generally ovoid in shape, but pointed at each end like a football for easier insertion. An endorectal balloon is about 1.5×4 inches (1-2×3-4 inches) and holds about 100 ml of fluid. However, other shapes may be desired for other purposes. A single groove or dimple positioned centrally may be ideal for prostate use, since this provides a depression into which the prostate can be wedged. Furthermore, shifting the depression proximally provides more material distally than proximally, allowing more stretch on inflation, thus providing a distal bulge to stabilize the seminal vesicles and prevent prostate motion in the distal direction.

When the balloon is intended for rectal use, it can also be advantageously provided with a gas lumen that travels the complete length of the balloon, protruding from the distal end and having openings past the distal end of the balloon, thus providing a passageway for the escape of gas. Ideally, such lumen has a smooth, rounded, closed soft tip with multiple side holes for gas entry, and is positioned centrally inside the balloon, although other positions and shapes are possible. In such cases, the fluid entry lumen for inflating the balloon need not traverse the length of the balloon, but only enter the balloon at the proximal end via, e.g., a low profile inlet fitment. Nested lumens, two lumens welded together, and bifurcated lumens can also be used, so long as there is fluid connection to the inside of the balloon, and a second fluid passageway traversing the balloon, but not in fluid connection with the balloon interior, such that gas can escape therethrough. A dedicated passageway can also be provided in the lumen for the sensor, but this is not needed, and the sensor can be positioned in the air provision pathway, or even outside the lumen altogether.

The balloon is preferably made of thermoplastic elastomers (TPE), especially thermoplastic polyurethane. Other balloon fabrication materials include latex, polyethylene (PE), polypropylene (PP), silicone, vinyl, polyvinyl chloride (PVC), low density polyethylene (LDPE), polyvinylidene chloride (PVDC), linear low density polyethylene (LLDPE), polyisobutene (PIB), and poly[ethylene-vinylacetate] (EVA) copolymers, nitrile, neoprene, and the like. It is also possible to use a laminar plastic, having more than one layer, e.g., a tougher interior layer and a biocompatible or slippery outer layer.

The ideal material is a translucent, biocompatible material, that has a durometer of less than 80-100 Shore A (ASTM D2240 or ISO 868), a tensile strength of at least 3000 psi (ISO 527-3 or ASTM D882-02), a 100% modulus of 500-1000 psi (ASTM D412), an elongation at break of at least 300% (ASTM D412), and that is air tight for 30-60 minutes even under 150% stretch. In some applications, the material should also be sterilizable, without loss of its qualities such as strength, etc.

One or a more fiducial markers are placed on a surface of the balloon and/or balloon distal tip. The fiducial markers can be affixed or formed on different surfaces of the balloon. One plurality of fiducial markers may be positioned on one side of the groove and a second set may be positioned on an opposite side of the groove. One set of fiducial markers may be positioned on the top surface of the balloon and a second set of fiducial markers may be placed on the bottom surface of the balloon.

Opaque markers can be letters indicating top (T) or right (R) and left (L) sides of the balloon, or numbers or any other shape, and can be particularly advantageous for those balloons whose shape is not radially symmetrical. An end marker can also be placed on the very tip of a gas lumen, if included therein.

A stopping means may be included therewith, and is a semispherical member that is slidably mounted on the shaft, which has a curved surface facing the balloon and a locking mechanism. The shaft can also have numerical or other indicia thereon for reproducible positioning. A gas lumen can also be provided for the balloon, wherein a separate air passageway extends beyond the distal end of the balloon, preferably have a soft, flexible closed tip and two or more side holes to allow gas escape.

In one embodiment, the stopper has an upper portion, generally smoothly rounded or semispherical, which fits snugly against the anus, and a hole or groove, through which the lumen(s) is/are threaded or fit. Other shapes may be used for other body cavities, and the stopper may be optional for other cavities.

A lower locking portion of the stopper snap locks against the lumen without blocking fluid entry, and preferably has interior fins or ridges lining its hole that engage the lumen, and prevent sliding, as a locking mechanism without such ridges is prone to do. Another means of making a locking stopper is to line the interior of the hole through which the lumens are threaded with a tacky material, so that friction locks the stopper in place. Another method is to make a portion of the interior compress the lumen enough to lock it in place, but not so much as to block the lumen. A conical interior may be beneficial for this. A hinge on the locking portion allows the lock to be opened, and the lock snap fits shut.

The details of the locking mechanism can be as shown in US2010145379, incorporated herein by reference in its entirety. The upper portion of the locking stopper has a groove reaching to the central hole, so that the stopper need not be threaded over the lumen, but this groove can be replaced with a hole and thus prevent stopper loss once the valves and luer lock are added to the end of the lumen. Of course, the central hole is not necessarily round as shown in US2010145379, especially if two lumens are welded together, but should reflect the cross section of the lumen(s).

The invention includes one or more of the following embodiments, and in any combination:

A immobilizing and sensing medical balloon, comprising:
a balloon having a fluid filling means;
a pocket on a surface of the balloon for holding a sensor or sensor cable.
A prostate immobilizing and sensing rectal balloon, comprising:
a flexible shaft having a fluid passageway extending therethrough and having a distal end and a
proximal end;
a balloon having an upper surface, a bottom surface, a distal end near the distal end of the shaft and
an proximal end that is affixed to the proximal end of the shaft, such that the fluid passageway
communicates with an interior of the balloon;
the upper surface comprising a conforming depression thereon, the lower surface being generally
rounded;
wherein the balloon has a non-inflated condition;
wherein the balloon has an inflated condition, wherein in the inflated condition the conforming
depression has depth and forms a central seating area that is configured to cradle a prostate when in
use; and
the balloon further comprising a pocket for holding a sensor cable, the sensor cable comprising:
a radiation sensor and cable for determining radiation dose, or
a motion sensor and cable for determining the motion of the balloon, or
both.
A prostate immobilizing and sensing rectal balloon, the rectal balloon comprising:
a flexible shaft having a fluid passageway extending therethrough and having a distal end and a
proximal end,
a balloon having an upper portion, a bottom portion, a distal end near the distal end of the shaft and a
proximal end that is affixed to the shaft, such that the fluid passageway communicates with an interior
of the balloon,
wherein the balloon comprises a top layer, a middle layer and a bottom layer, the layers bonded
together along their edges to form the balloon,
wherein the middle layer is connected to the top layer to form a conforming depression,
wherein the balloon has a non-inflated position,
wherein the balloon has an inflated position wherein the conforming depression engaging and
immobilize a prostate in use;
a pocket on the middle layer for holding a sensor cable (the sensor and/or sensor cable as described
herein);
The pocket can co-located with the conforming depression, to either side, or both. Pockets can be on
an outer surface, and inner surface, or on a middle layer if a 3 or more layer balloon. A sensor can be
in the pocket, or the balloon with pocket can be sold separately from the sensor.
The radiation sensor can comprise a plastic scintillator fiber optically coupled to an optical cable
operatively coupled to an adaptor for reversible coupling to a separate scintillation detection and
display unit.
The motion sensor can comprise a electromagnetic motion sensor comprising coils operatively
coupled to an adaptor for reversibly coupling to a separate motion detection and display unit.
The sensors can be bundled together (provided their mechanisms of action do not interfere) or
separate, and housed in separate pockets.
The pocket can be on an inner surface of the balloon, and the sensor cable can run through the shaft
and out an opening therein and into the pocket. The pocket could also be on an upper surface and the
sensor cable runs along the shaft and into the pocket, and an attachment means reversibly couple the
sensor cable to the shaft.
The balloon can also include one or more fiducial markers thereon. Fiducial markers can be a radio-
opaque material or radiodense material, including titanium, tungsten, barium sulphate, bismuth,
iodine, and the like
A balloon can also include a stopping means comprising a semispherical member slidably mounted on
the shaft, the semispherical member having a curved surface facing the balloon and a locking
mechanism to lock the stopping means at a desired location on the shaft.
A balloon can also comprise a gas lumen, with a separate fluid path extending beyond the distal end
of the balloon. The gas lumen tip preferably has a closed tip, with holes on the sides for gas entry.
The tip can also include a radiopaque marker.
Method of treating a prostate are also provide, one method comprising:
inserting a prostate immobilizing rectal balloon having a conforming depression and a plastic-scintillator
radiation sensor thereon into a rectum of a patient;
inflating the balloon such that a prostate engages with the conforming depression;
treating the prostate with external beam radiation therapy;
assessing a radiation dosage via the plastic-scintillator radiation sensor; and
adapting radiation therapy plans when radiation dosage data has been acquired.
Another method of treating a prostate, comprising:
inserting a prostate immobilizing rectal balloon having a conforming depression and an
electromagnetic motion sensor thereon into a rectum of a patient;
inflating the balloon such that a prostate engages with the conforming depression;
treating a radiation target area at the prostate with external beam radiation therapy;
assessing a motion of the prostate, rectum or balloon via the electromagnetic motion sensor; and
adapting radiation therapy plans when the prostate moves away from the radiation target area.
A alternative method of treating a prostate, comprising:
inserting a prostate immobilizing rectal balloon having a conforming depression and a plastic-
scintillator radiation sensor and a an electromagnetic motion sensor thereon into a rectum of a patient,
inflating the balloon such that a prostate engages with the conforming depression;
treating a radiation target area at the prostate with external beam radiation therapy;
assessing a radiation dosage via the plastic-scintillator radiation sensor and adapting radiation therapy
plans when a radiation dosage data has been acquired; and
assessing a motion of the prostate or the balloon or both via the electromagnetic motion sensor, and
adapting radiation therapy plans when the prostate moves away from the radiation target area.
Yet another method of treating a prostate, comprising:
inserting a prostate immobilizing rectal balloon having a pocket therewith containing a sensor cable
into a rectum of a patient;
the sensor cable comprising:
a plastic-scintillator radiation sensor, or
a an electromagnetic motion sensor, or
both (1) and (2);
inflating the balloon such that a prostate engages with the balloon;
treating a radiation target area on the prostate with radiation; and
assessing a radiation dosage via the plastic-scintillator radiation sensor and adapting radiation
treatment plans when radiation dosage data been acquired; or
assessing a motion of the prostate, rectum or the balloon via the electromagnetic motion sensor, and
adapting radiation treatment when the prostate moves away from the radiation target area; or
both (1) and (2).

The term “distal” as used herein is the end of the balloon inserted into the body cavity, while “proximal” is opposite thereto (e.g., close to the medical practitioner). The terms top and bottom are in reference to the figures only, and do not necessarily imply an orientation on usage. The length of balloon and lumen is the longitudinal axis, while a horizontal axis and vertical axis cross the longitudinal axis.

By “weld” herein we mean any method of attaching two layers of polymeric film together. Thus, the welds or attachment points can be glued, heat welded, RF welded, ultrasound welded, solvent welded, hot gas welded, freehand welded, speed tip welded, extrusion welded, contact welded, hot plate welded, high frequency welded, injection welded, friction welded, spin welded, laser welded, impulse welded or any other means known in the art.

By “central” portions herein, we are distinguishing from the edges in a bilayer construction. Thus, central refers to portions inside the edges, but an exactly central position is not implied.

By “pinch” what is meant herein is that a balloon surface is folded at a small area, creating a portion where the balloon is bilayered. In other words, the surface is bent and the two surfaces on either side of the bend brought together so as to be juxtaposed or directly adjacent. This pinch can be glued or otherwise welded, making the bilayer structure permanent. Outside of the pinch area, the balloon has the usual single layer structure.

By “fold inside” or “pinch inside” or “folded internally” or any similar phrases, what is mean is that the material is folded such that the outer surfaces of the balloon are in juxtaposition, and so that the bilayer portion is “inside” the balloon.

By “conforming depression” what is meant is that the depression is retained even on hyperinflation or squeezing or otherwise constraining the balloon. Thus, the balloon holds its shape, even in the compressed, slippery, mobile environment inside the rectum, and will tend to continue to cradle the prostate, as opposed to letting it slide off the balloon surface.

By “baffle” what is a meant is a small strip of material of length less than the expanded width between the two surfaces to which it is welded. The baffle is thus welded to one or more surfaces of the balloon and/or the lumen, and serves to control the depth of a conforming depression, longer baffles leading to shallower depressions, shorter baffles leading to deeper depressions. The pinch described above, serves the same function as the baffle, but is not a separate piece of material, but made directly from the balloon surface material.

By “groove” what is meant is a depression that is longer than its width. By “dimple” what is meant is a depression that is about as long as its width.

By “pocket” herein what is meant is a small channel or tunnel or tube to enclose (preferably on 3 sides) the one or more sensors provided with the balloon. For a rectal balloon the pocket is preferably on the surface of the balloon that cradles the prostate and preferably coincides with the groove or dimple or other conforming depression. The pocket can be on the inner surface, allowing the sensor to be threaded through the lumen and into the pocket, but this is not essential and the pocket also be on the outer surface. For a reusable sensor this may be a better location, allowing the user to easily slip the sensor into the pocket in use, and remove it for sterilization after use (if needed).

A “plastic-scintillator radiation sensor” generally comprises a plastic scintillator optically couple to a fiber optic cable operatively coupled to an adaptor or connector, wherein the entire sensor is encased in an opaque jacket or otherwise protected from ambient light. The remaining portions of the system, e.g. detector, display unit, processors and the like are generally sold separately from the sensor cable, and are well known in the art and not detailed herein. The remaining portions of the system, e.g. detector and display unit, processors and the like are generally sold separately from the sensor cable, and are well known in the art and not detailed herein.

A “electromagnetic motion sensor” as used herein generally refers a sensor having 2 or 3 coils therein, which produce an electrical current in a variable magnetic field in which the motion sensors are located. These are electrically coupled to an adaptor or connector and the entire cable is electrically insulted. The remaining portions of the system, e.g. EM field generator, amplifier units (if any), display unit, processors and the like are well known in the art and not detailed herein. In one embodiment the motion sensors used herein utilize electromagnetic fields to determine motion thereof. Electromagnetic navigation systems are generally based on the Biot Savart law, the principle that in the presence of a known magnetic field generator, the magnetic field vector in a given location can be measured in terms of magnitude, direction, length, and proximity of the current generating the field by a sensor. Generally the motion sensor includes a transmitter assembly and a sensor assembly. The transmitters are typically in the form of coils, and mutually orthogonal relative to each other. The sensor assembly may have one or more sensors and capable of monitoring the magnetic fields generated by the transmitter assembly. The individual sensors may be coils, flexgate transducers, magneto-resistive sensors, Hall effect sensors or any other devices capable of providing precision measurements of magnetic fields. In practice, a small electromagnetic field generator in the form of a small, block-like device creates a small, differential magnetic field into which a sensor coil may be placed. This small field is typically only 50×50×50 cm, but can be larger or smaller for different applications. The coils detect the rapidly changing magnetic field, and per Faraday's law of electromagnetic induction, elicit a weak electrical current. It is the processing of this current within the magnetic field that allow delineation of the sensor, and thus, balloon position, within the confined space.

As an alternative, the sensors described herein can be wireless, in which case the pocket can be sealed completely around the sensor, providing a waterproof environment. However, the currently preferred sensors are wired, and thus include a cable and adaptor for connection to separate detector units.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, and “include” (and their variants) are open-ended linking verbs and allow the addition of other elements when used in a claim. The phrase “consisting of” excludes additional elements, and the term “consisting essentially of” excludes material elements, but allows the inclusion of nonmaterial elements, such as labels, instructions for use, radio-opaque markers, stoppers, and the like.

### BRIEF DESCRIPTION OF DRAWINGS

FIG. 1 is a side elevational view, partially transparent, which shows the rectal balloon apparatus in an un-inflated condition.

FIG. 2 is a side elevational view of the rectal balloon apparatus of the present invention in an inflated condition.

FIG. 3 is an isolated view showing the compact folding of the balloon over the end of the shaft.

FIG. 4 is a top view of the inflated balloon as used in the rectal balloon apparatus of the present invention showing, in particular, the application of fiducial markers to a surface of the balloon and a sensor in the groove.

FIG. 5 is a side view, partially transparent, of the balloon of the rectal balloon apparatus in a first inflated condition.

FIG. 6 is a side view, partially transparent, of the balloon of the rectal balloon apparatus in the second inflated condition.

FIG. 7 is a view of the operation of the stopper of the rectal balloon apparatus.

FIG. 8 is side view of the balloon of the rectal balloon apparatus positioned within the rectum and in an inflated condition.

FIG. 9 is a cross-sectional side view of the balloon of the rectal balloon apparatus showing the plurality of layers that form the balloon, and groove formed by attaching the top surface to the middle layer.

FIG. 10 A-E shows various ways of making a conforming balloon. In general, the outer surface of the balloon must be restrained from free expansion, and this can be done by welding it to an inner layer (10A), to itself or to the lumen (10B-D) (or both as shown), Alternatively one or two small baffles can be used to connect a layer to the lumen (10E, two baffles shown). FIG. 10C also shows a gas lumen.

FIG. 11A is a cross-sectional top view of the balloon of the rectal balloon apparatus, and FIG. 11B is a cross-sectional side view of the balloon of the rectal balloon apparatus.

FIG. 12A-B shows the different four-layer configuration of the balloons.

FIG. 13 is a cross section of a balloon wherein the weld between the top and middle layer is U-shaped, providing a central pocket into which the cable sensor can fit. This avoids the use of a fourth layer to make the pocket. The sensor (not shown) travels inside the lumen, out a nearby exit hole and into this interior pocket

FIG. 14A-B shows a balloon where the pocket is made by a fourth layer on the outer surface of the balloon. This design would be suitable for a reusable sensor, allowing the sensor to be sterilized and reused with a new balloon. In FIG. 14A the cross section shows two pockets, on each side of the central weld, but it could easily be a single exterior pocket positioned centrally. FIG. 14B shows the same balloon in perspective, with a bifurcated sensor cable fitting into each pocket, and the proximal end of the cable is thus fitted with clips for secure attachment to the proximal end of the lumen.

FIG. 15A is a perspective of the assembled radiation sensor cable that used in the endorectal balloon. FIG. 15B is a cross section view showing the details of the radiation sensor.

### DETAILED DESCRIPTION OF THE INVENTION

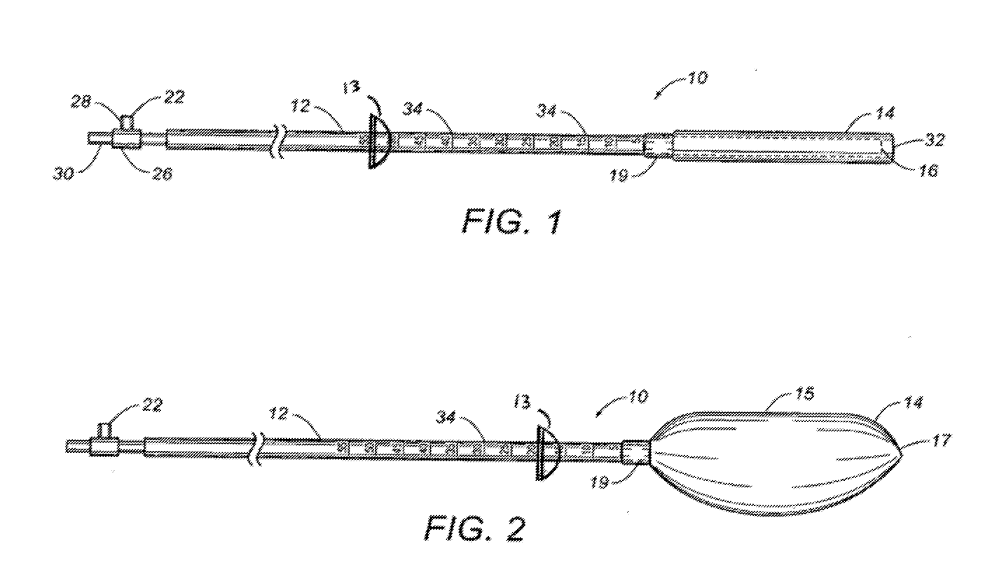

Referring to FIG. 1, there is shown a rectal balloon apparatus 10. The rectal balloon apparatus 10 includes a shaft or lumen 12 having a fluid passageway extending therethrough. A balloon 14 is affixed over the end 16 of the shaft 12. The balloon 14 is shown in an un-inflated condition. The fluid passageway of the shaft 12 can communicate with the interior of the balloon 14. Also shown is the stopper 13, which is slidable along the shaft 12. The stopper 13 has a hemispherical shape, the rounded end facing distally (toward the balloon). The stopper 13 serves to assure uniformity in the positioning of the balloon 14 during radiation therapy, and the rounded surface provides comfort to the patient.

The shaft 12 is a generally longitudinal shaft and has a fluid passageway extending through the center thereof. The shaft 12 is made of a flexible material, and can bend slightly to conform to the rectum and provide comfort, but still be stiff enough to be inserted thereinto.

A valve assembly 22 is affixed to the shaft 12 opposite the balloon 14. The valve assembly 22 can have a variety of configurations. FIG. 1 illustrates the valve assembly 22 as an inline valve assembly configuration. The valve assembly 22 may also be an angled valve assembly configuration. The valve assembly 22 includes a stopcock 26. A valve 28 facilitates the ability of the stopcock 26 to open and close so as to selectively allow the fluid to pass into the shaft 12. A port 30 allows the valve assembly 22 to be connected to a supply of the fluid. When the stopcock 26 is opened by the rotation of the valve 28, the fluid will flow through the valve assembly 22, through the interior passageway of the shaft 12 and into the interior of the balloon 14. The valve 28 can then be closed so as to maintain the inflated configuration of the balloon 14. When the procedure is finished and the fluid needs to be removed from the balloon 14, the valve 28 of stopcock 26 can then be opened so as to allow for the release of fluid therethrough.

The opposite end 16 of the shaft 12 contacts the end 32 of the balloon 14. The end 16 is preferably curved or dome-shaped so as facilitate the introduction of the balloon 14 into the rectum. The shaft 12 has numerical or other indicia 34 formed therealong. These numerical references are indicative of the distance that the balloon 14 has been inserted into the rectum. As such, the indicia 34 provide a clear indication to the medical personnel of the desired location of the rectal balloon 14. Here, the stopper is shown positioned at indicia 34 number “55.”

A ring 19 is affixed to the shaft 12 adjacent to the balloon 14. This ring 19 can be of a bright color, such as blue, so as to provide the medical personnel with positive indication of when the balloon 14 is past the anal verge. The ring 19 is approximately 5 millimeters long. The stopper 13 is shown as positioned away from the balloon 14. This would be the position prior to insertion. The stopper 13 is slidably mounted on the shaft 12. The stopper 13 has a semi-spherical shape so as to conform to the entrance of the rectum. A suitable locking mechanism can be provided so as to fix the stopper at a desired location.

FIG. 2 illustrates an isolated view of the apparatus 10 after being installed within the rectum. The fluid (e.g., 100 ml or air or water or saline) can be introduced through the valve assembly 22 and through the interior passageway of the shaft 12 so as to inflate the balloon 14. The ring 19 is shown as adjacent an end of the balloon 14. The balloon 14 has a seating area 15 so that the prostate can be properly positioned thereon. The balloon 14 has a head portion 17 adjacent the tip of the balloon 14 opposite the shaft 12. When the balloon 14 is installed and inflated, the prostate will reside on the flat surface 15 in a seated position. The head portion 17 will abut the tip of the prostate.

After the procedure has been completed, the balloon 14 can be deflated and easily pulled outwardly of the rectum in its deflated condition. In FIG. 2, it can be seen that the stopper 13 has been moved along the shaft 12 (from its position in FIG. 1) to indicia 34, specifically at the number “20.” This serves to assure that the balloon 14 will be in a proper position during subsequent radiation treatments. The numbers can be noted in the patient record for use with new balloons.

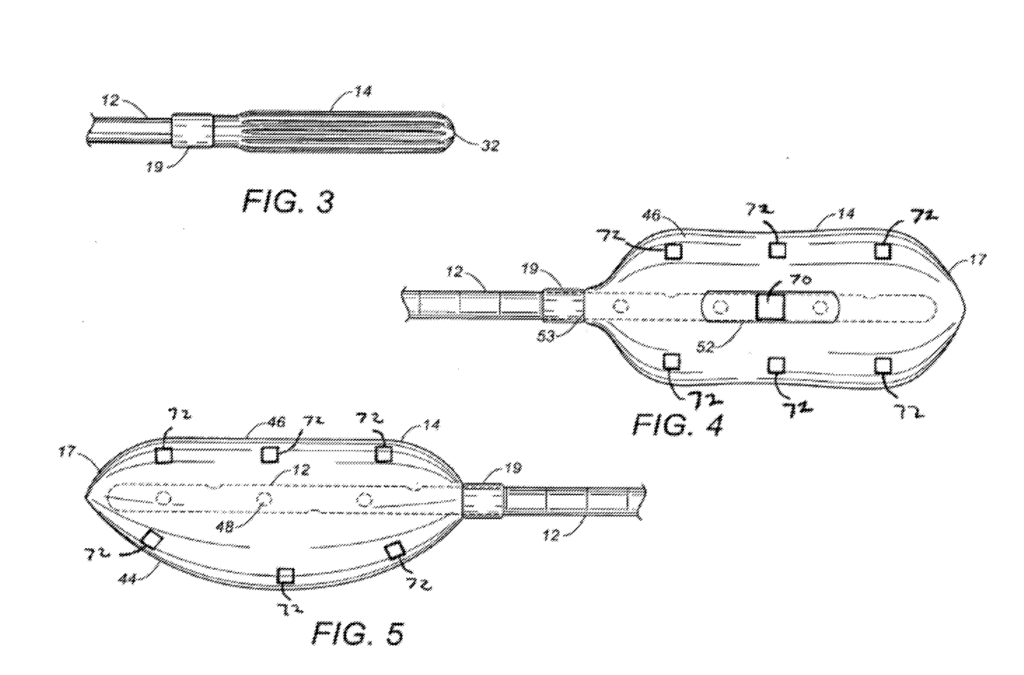

FIG. 3 shows that the balloon 14 is neatly folded and compressed over the outer diameter of the shaft 12. The shaft 12 will have a rounded end abutting the end 32 of the balloon 14. As such, a comfortable rounded profile is provided at this end 32. The end 32 of the balloon 14 is sealed over the outer diameter of the shaft 12. The balloon 14 is pre-vacuumed during production to produce a minimal profile during use. The ring 19 is placed over the shaft 12.

FIG. 4 is a top view of the balloon 14 from the side of the balloon 14, which engages with the prostate. Central seating area 46 is shown as having a groove 52 formed thereon. The groove 52 is generally rectangularly-shaped (with rounded corners) and engages with the tip of the prostate, reducing lateral motion. The central seating area 46 and the groove 52 greatly enhance the holding stability of the balloon 14 of the present invention. In FIG. 4, it can also be seen that head portion 17 of the balloon 14 is generally V-shaped. This shape makes insertion of the balloon 14 into the rectum easier for medical personnel and more comfortable for the patient. The balloon 14 has a thermally welded bond 53 connecting it to the shaft 12.

Importantly, in FIG. 4 it can be seen that a sensor 70 is located within the groove 52 of the central seating area 46. The sensor 70 allows the treating physician to determine the dose of radiation being received at the treatment area when the balloon 14 is in place. The sensor 70 is located in the middle of the groove 52. This location is ideally centrally located on the prostate when the balloon 14 is in place. By positioning the sensor 70 adjacent the prostate, an accurate measurement of the radiation delivered to the prostate is achieved.

The sensor 70 can be chosen from any of the available implantable sensors that enable user to monitor the radiation dosage for external beam radiation therapy devices. A particularly preferred sensor is the sensor described in 61/481,503, filed May 2, 2011, and the utility filing related thereto Ser. No. 13/444,584, filed Apr. 11, 2012, and expressly incorporated by reference herein in their entirety. That sensor is a plastic scintillator detector cable comprising a single, short length of scintillator fiber operably coupled to a suitable length of optic fiber, which has a standard data coupler or connector at the end of the cable opposite the scintillator fiber. The scintillator detector is thus at the distal end of the cable and a suitable data coupler is at the proximal end, and the entirety of the cable is enclosed in a flexible, opaque covering (e.g., the typical wire jacket).

In another embodiment, the cable has at least two separate, but closely juxtaposed, plastic scintillator detectors. The two detectors are parallel, but offset from one another in the longitudinal axis, so that radiation can be simultaneous assessed at two ends of a target, such as on either end of the prostrate or both ends of an irradiated throat area, and the like.

In preferred embodiments, this sensor cable is contained in the layer between the upper and middle layers of the balloon, thus being protected from the environment and immediately adjacent the prostate, and the distal end of the cable affixed to at least a portion of the shaft such that the connectors extend outside the body cavity and can be plugged into the appropriate device (e.g., a scintillation counter).

FIG. 4 also shows a plurality of fiducial markers 72 located on or below the surface of the balloon 14. The fiducial markers 72 may be made of a tungsten material or any of the known radiopaque or reflective materials, depending on the imaging means used. Our experimentation has shown that through the use of these fiducial markers 72 on the balloon 14, a treating physician can get a very clear image of the anterior and posterior walls of the rectum. In FIG. 4, it can be seen that the fiducial markers 72 are positioned in spaced relation to each other on the top surface of the balloon 14. Three of the fiducial markers 72 are positioned in linear alignment on one side of the groove 52. Another three fiducial markers 72 are arranged on the opposite side of the groove 52.

A further benefit can be realized by utilizing an additional fiducial marker in the form of a radioactive seed implanted or injected into the prostate. The radioactive seed combined with the fiducial markers 72 allows for triangulation to make certain that the balloon is in the correct position for treatment.

Additional benefit can be realized if the fiducial marker is contained on or within the cable. For example, the fiducial marker can be at the tip or on the surface of the cable, and in fact, the fiducial marker can be positioned inside the cap designed in Ser. No. 13/444,584. It could also be placed on or inside the tip of the balloon shaft.

FIG. 5 is an isolated view of the balloon 14 as inflated to a first inflated condition. In this condition, the balloon 14 has a central seating portion 46, a head portion 17 and a bottom portion 44. When inflated, the central seating area 46 has a lateral flatness for the prostate to rest upon. The lateral flatness of the seating area 46 (together with groove 52) will prevent the prostate from sliding to one side or the other. The bottom portion 44 is rounded and contacts the rectal wall. The head portion 17 is generally V-shaped so as to facilitate easier insertion of the balloon 14. The material of the balloon 14 is formed of a non-latex material so as to avoid allergic reactions. The shaft 12 is shown extending into the interior of the balloon 12.

A plurality of holes 48 are formed in the shaft 12 through which the balloon 14 is filled with fluid. The plurality of holes 48 are formed within the balloon 14 so as to allow fluid to be introduced into and removed from the balloon 14. It can be seem that each of the holes 48 is spaced from and offset by 90° from an adjacent hole around the diameter of shaft 12. A total of six holes are formed in the shaft 12 within balloon 14 so as to allow the fluid to pass from an interior of shaft 12 to the interior of the balloon 14. This arrangement of holes 48 facilitates complete extraction of the fluid from the balloon 14. Under certain circumstances, one of the holes may become clogged or blocked by contact between the body and the balloon, the staggered arrangement of holes assures that the unblocked holes 48 allow the fluid to continue to be easily extracted.

In FIG. 5, it can be seen that additional fiducial markers 72 are positioned on the opposite side of balloon 14. The fiducial markers 72 are generally arranged symmetrically on opposite sides of the balloon 14.

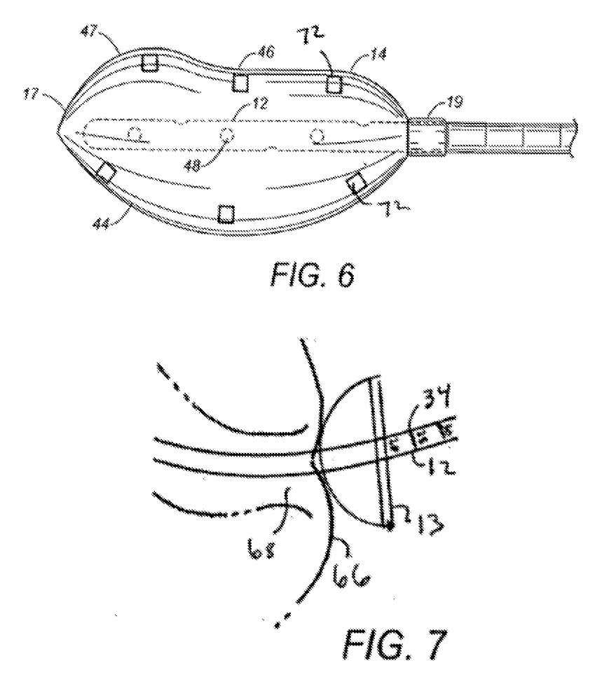

FIG. 6 is an isolated view of the balloon 14 as inflated to a second (more) inflated condition. In the second inflated condition, the balloon 14 has a first bulge 47 formed at the head portion 17 (proximal end). The balloon also has a laterally flat seating portion 46. The first bulge 47 can be utilized in certain conditions to better isolate the prostate. Generally, the first bulge 47 will be introduced when at least 110 ml of fluid are introduced into the balloon 14, so as to slightly overinflate the balloon.

FIG. 7 shows an isolated view showing the stopper means 13 when the balloon 14 has been inserted into the patient's rectum. The stopper means 13 has been moved along the shaft 12 up against the patient's buttocks 66 and adjacent the anus, without having entered the anal canal 68. It can be seen that the stopper means 13 is positioned such that it resides along indicia 34 number “20.” Thus, during a first treatment, a treating physician would place the balloon 14 in the proper position and then slide the stopper means 13 up against the patient's buttocks 66. The physician would then make note of the position of the stopper means 13. Then, during subsequent treatments, it would be easier for the physician to place the balloon 14 properly. The physician would simply have to insert the balloon 14 and shaft 12 to the extent necessary such that the stopper means 13 would rest at the same indicia 34 as during the previous treatment when the stopper means 13 is pushed up against the patient's buttocks 66. The stopper means may be shaped in a variety of ways, but it is shown here to have an arcuate front surface to conform to a patient's anatomy.

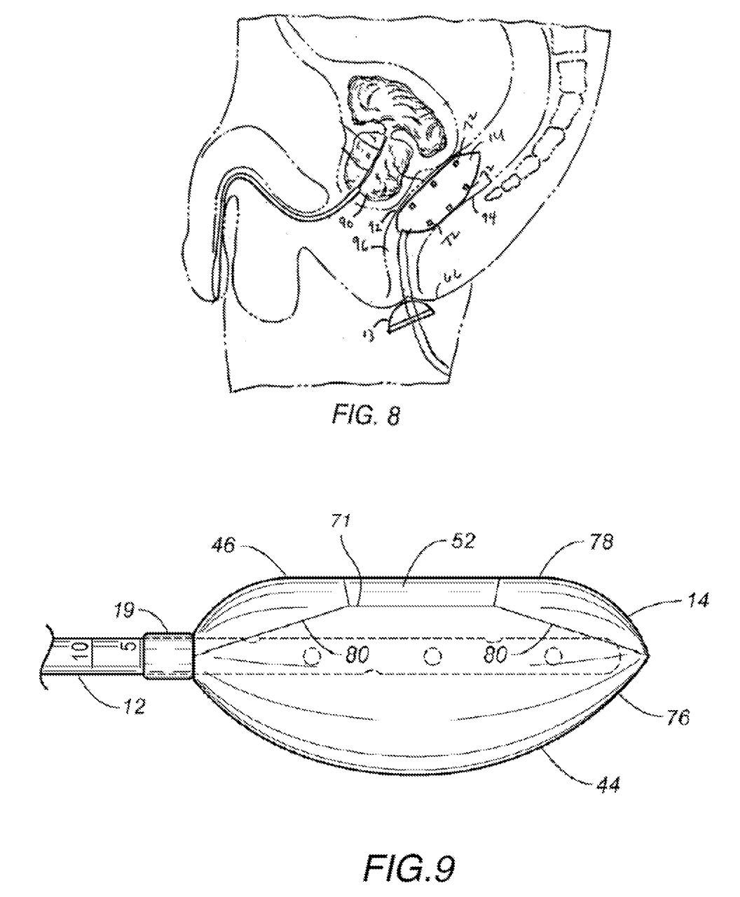

FIG. 8 shows an anatomical side view of the rectal balloon apparatus 10 positioned within a patient's rectum. The balloon 14 is shown in an inflated condition and positioned up against and between the anterior wall 92 and the posterior wall 94 of the rectum 96. It can be seen that the balloon 14 is positioned adjacent the prostate 90. Additionally, it can be seen that the plurality of fiducial markers 72 are generally positioned adjacent either the anterior wall 92 or the posterior wall 94 of the rectum 96. Thus, when a treating physician can determine the position of the plurality of fiducial markers 72, he or she may obtain a clear image of the contours of the anterior wall 92 and the posterior wall 94 of the rectum 96 by essentially “connecting the dots.” FIG. 8 also shows the importance of the flexible aspect of the shaft 12 and the utilization of the stopper means 13.

FIG. 9 is a cross-sectional side view of the balloon 14, showing the plurality of layers that form the balloon 14. A bottom layer 76 forms the bottom portion 44 of the balloon 14. A top layer 78 forms the upper portion, including central seating area 46 and the groove 52, of the balloon 14. A middle layer 80 extends between the bottom layer 76 and the top layer 78. The middle layer 80 is connected to the top layer 78 at the groove 52.

As discussed above, the groove 52 at the central seating area 46 engages with the tip of the prostate to reduce the lateral movement of the balloon. To achieve that, however, it is important that the groove 52 maintains its shape even when the balloon 14 is subject to external pressure when put inside a patient's rectum. The groove 52 is thus formed by welding or otherwise attaching the top layer 78 with the middle layer 80 at the groove bottom 71. This way, a recessed area 52 with some depth can be maintained, thus its engagement with the patient's prostate, regardless of the external pressure that may or may not cause the remainder of the balloon to deform. A skilled artisan can understand that the bonding between the top layer 78 and the middle layer 80 at the groove bottom 71 can be achieved by other equivalent methods known in the field.

In general, the present invention assures uniformity and reproducibility of positioning. The stopper 13 provides an initial indication of the depth of positioning of the balloon 14. It is possible that the balloon 14 could have an improper rotational position in the rectum. A proper orientation of the balloon 14 is achieved by viewing the fiducial markers 72 by any imaging system. The lateral flatness of the balloon 14 is assuredly positioned against the prostate. In essence, the prostate is wedged by the inflated balloon into the dimple created by the groove 52, and is unable to slip from one side to the other as in the prior art non-conforming balloons. The sensor 70 is thereby properly positioned at the same location during all treatments. The sensor 70 can then be used to accurately determine the amount of radiation delivered during each external beam radiation treatment.

In use, the sensor cable is outfitted with adaptors for connection to the requisite radiation detector instrumentation, such as CCD camera, photodetector, photomultiplier tube, scintillation counter, MOSFET, vacuum photodetector, microchannel plates, and the like, which operably connects with a processor having the needed software to assess and report radiation dose.

Using the rectal balloon with fiducial markers and radiation sensor described herein, the radiologist can accurately position the balloon, wedge the prostate into the groove by inflation, and determine exactly where the device is using a variety of imaging means. Further, the radiologist can accurately measure radiation dose at multiple locations on the prostate, thus allowing further refinements in dosimetry.

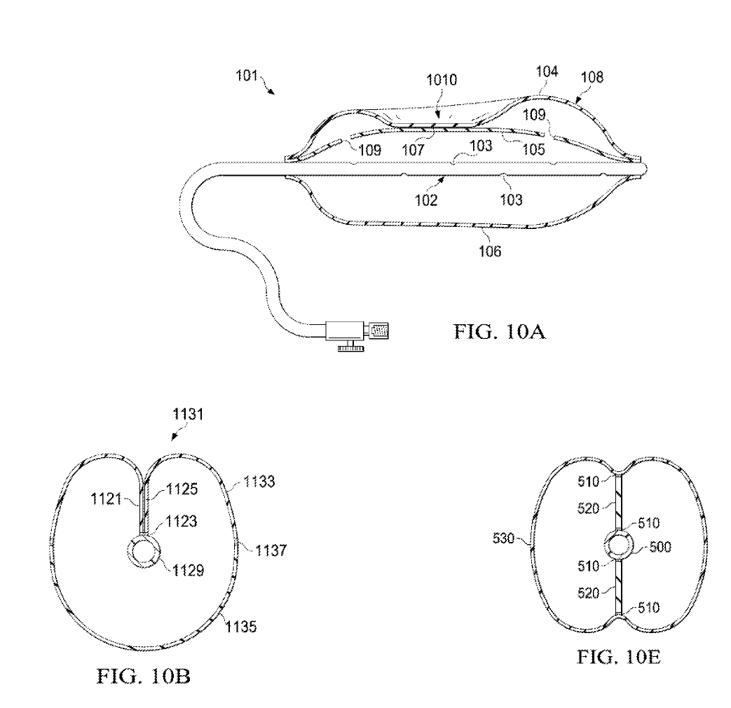

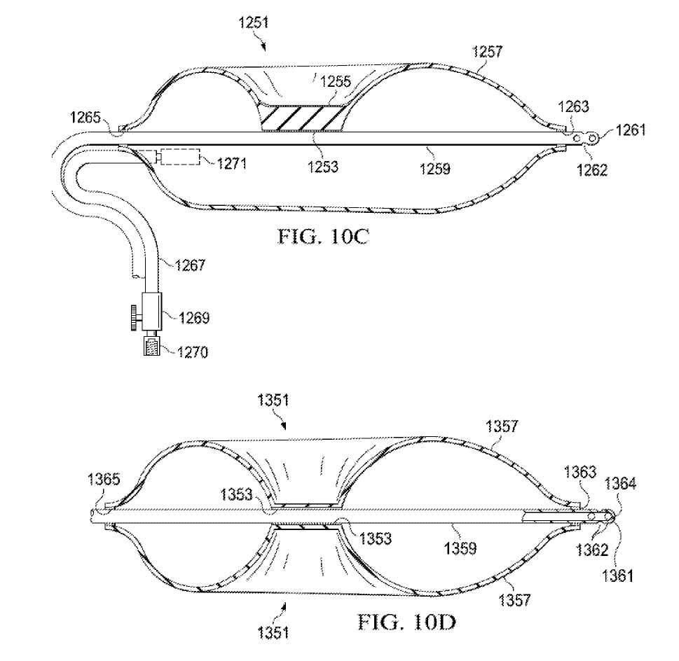

FIG. 10A-E illustrate a variety of methods for making a conforming balloon. FIG. 10A the balloon is a single chambered balloon, albeit being made of three layers. Thus, the middle layer of balloon material has perforations or gaps so that the balloon consisted of a single fluid chamber and the entire device could be filled with a single lumen. This is shown in FIG. 10A, which is a cross section of the prior art rectal balloon 101 with lumen 102 having offset holes 103 for fluidic communication with the interior of the balloon. The use of a plurality of offset holes is generally preferred because it helps to prevent inadvertent hole blockage e.g., by the balloon material or the rectal walls, thus ensuring easy fluid flow.

This balloon has a top layer 104, a middle layer 105, and a bottom layer 106, which are welded together along the outer edges (not shown), and also affixed to the lumen, in this case at both the distal and proximal ends. The top layer 104 is welded 107 to the middle layer 105 along the central line of the balloon, but shifted proximately, so that the distal portion of the balloon bulges 108 more than the proximal portion on hyperinflation. The middle layer also has holes or gaps 109 so that the balloon comprises only a single fluid chamber and thus needed only a single fill means, but dual fluid filling means could be provided for a two chamber balloon (see e.g., US20130123621). The balloon filling means (typically a lumen, stock cock and luer connector) are not labeled in this figure, but are typical in the art.

The weld 107 of top layer 104 to middle layer 105 provides a groove 1010 (or indent or depression) having some depth into which the prostate can be wedged, and this grooved depression is retained on inflation, and even on hyperinflation, or in the constrained environment of the rectum. Although a groove 1010 is shown, a dimple could also suffice, and the weld could be made shorter. The physical coupling of the middle baffle layer to the top layer provides a physical restraint against expansion or stretching, and the balloon is conforming—that is it holds its shape even in the highly mobile constrained environment of the rectum.

We now show how to make a similar conforming shaped balloon using a unitary or binary balloon construction and fewer welds.

A unitary balloon is made by any conventional method and in any desired shape. For example, a tubular form is heated, immersed in a tank of coagulant solution for a few seconds, heated again and then immersed in a tank of latex. The coagulant causes the latex to coat the form, and the longer the forms are left in the tank, the thicker the coating that sticks to them. The forms must be inserted and removed at carefully controlled speeds to avoid trapping air bubbles and to achieve an even, thin coating. The coated forms are then immersed in a tank of leaching solution (often plain water) to dissolve and leach away excess coagulant, and the rubber or polymer on the forms is dried and cured as needed. The balloons are then mechanically removed from the forms, e.g., with a spray of water or air.

Whether the balloon is unitary or binary (two layers), the balloon can then be shaped to make a conforming depression, as shown in FIG. 10B. For example, a spot of glue is laid on the balloon's outer surface, and the balloon pinched at that spot to form a welded pinch 1121. Alternatively, the pinch or fold 1121 can be made first, and then welded or glued 1125 to form the welded fold. This can be done with a jig that fits inside the balloon and folds it. In yet a third alternative, the pinch can be omitted, and the upper layer simply welded to a central lumen. In a fourth embodiment, both top and bottom layers can be welded to a lumen.

The lumen 1129 is also coated with a spot of glue and inserted into the balloon, such that the pinch 1121 is then welded 1123 to the lumen. This can also be done with jigs to hold the balloon and lumen. The balloon is welded to at least the distal end of the lumen, preferably both ends, valve means are provided and if needed the balloon is sterilized before packaging. The position of the lumen and depth of groove can be influenced by changing the amount or depth of balloon pinch (-d-), a smaller pinch weld moving the lumen closer to the edge of the balloon and making the groove more shallow.

Although we describe a unitary balloon, it is also possible to make the shaped balloon in two layers. See e.g., optional edge weld 1137. In some cases the two-layer construction may make the pinch/lumen welds easier, especially where the balloon is quite small and it is difficult to create a weld inside a unitary balloon. The balloon is as described above, but an additional weld 1137 is shown at the outer edges of the two layers 1133 and 1135. The use of two layers also means that the two layers can be made of different materials, e.g., a less stretchy or thicker material on one side that will not stretch as much and thus provide a flatter surface. When the device is welded, it can be inverted so as to put the edge welds, which can be stiff or sharp, on the inside of the balloon if needed.

A rectal balloon 1257 is shown in cross section along its longitudinal axis in FIG. 10C. Here a gas lumen 1259 traverses the balloon and is fitted with a soft rounded and closed tip 1261 having offset holes 1262 for gas entry. The balloon is fitted at the proximal end with a low profile inlet fitment 1271 and lumen 1267 with valve means 1269 and luer connector 1270. As an alternative arrangement, the fluid input lumen can be alongside the gas lumen, or the gas lumen can be nested inside the fluid lumen.

The pinch weld is shown at 1255, and the weld to the lumen 1253 is shown in black. Additional welds 1263 and 1265 are to the distal and proximal ends of gas lumen 1259. The depression or groove 1251 is thus clearly seen. On hyperinflation, the distal end of balloon 1257 will bulge distally of the groove 1251 (not shown) since there is more material here, and thus, there will be more stretch.

FIG. 10D is another variation where the pinches are omitted entirely, and both layers are welded 1353 to the lumen 1359, creating a pair of deep grooves. Groove depth can be decreased on one or both sides by combining a baffle with the lumen weld, which allows the surface of the balloon to get farther away from the lumen. If one of these lumen welds is omitted, the balloon would be suitable for use in immobilizing the prostate.

In yet another variation, the pinch can be replaced with a baffle that is a small piece or strip of film welded at both the top layer and the lumen, wherein the width of the baffle controls the depth of the groove. FIG. 10E shows a variation, wherein there are two baffles 520 that are each welded 510 to the unitary balloon 530 and to the lumen 500. However, a single baffle can be used, and the baffle can attach to the lumen and balloon where a single depression is needed.

Using the pinch weld, lumen welds and layer to layer welds as described herein, it is possible to make a shaped balloon with one or more conforming depressions anywhere on its surface. Further, bulges can be created with thinner or more elastic material, or shaped on a unitary balloon mold, or cut in a two layer balloon outline, as desired. Thus, using the principles described herein, a variety of conforming shapes are possible.

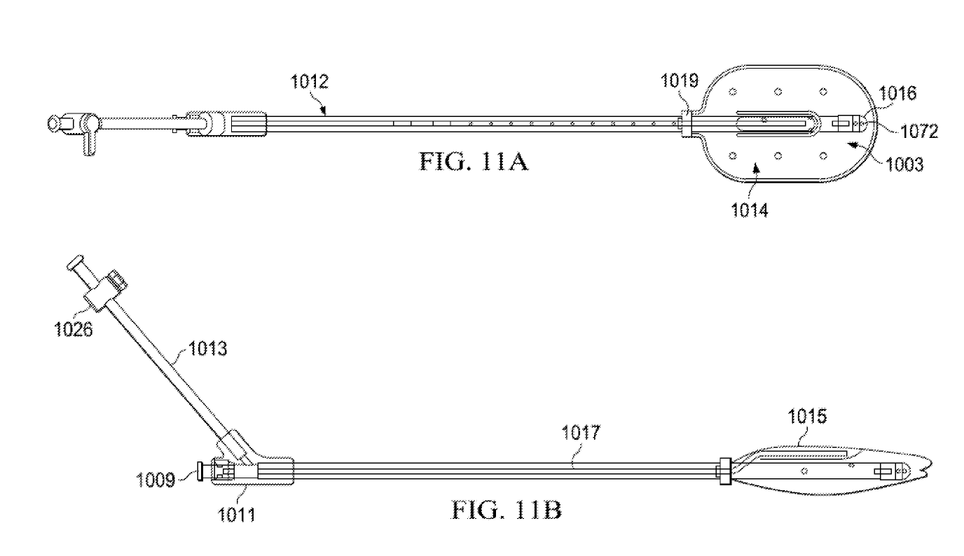

FIG. 11A is a cross-sectional top view of the balloon of another embodiment, whereas FIG. 11B shows a cross-sectional side view of the balloon. In FIG. 11A, the rectal balloon apparatus 1003 has a hollow shaft 1012 that extends inside the balloon 1014. At the balloon end of the shaft 1012 there is provided a soft tip 1016 and a fiducial marker 1072. Turning to FIG. 11B, a flat seating area 1015 is provided for contacting the prostate gland during treatment. A bifurcated hub 1011 is provided, with a port 1009 extending to the hollow shaft. A tunnel 1017 extends from the port 1009 at the hub 1011 through the hollow shaft 1012 and rises into the seating area 1015.

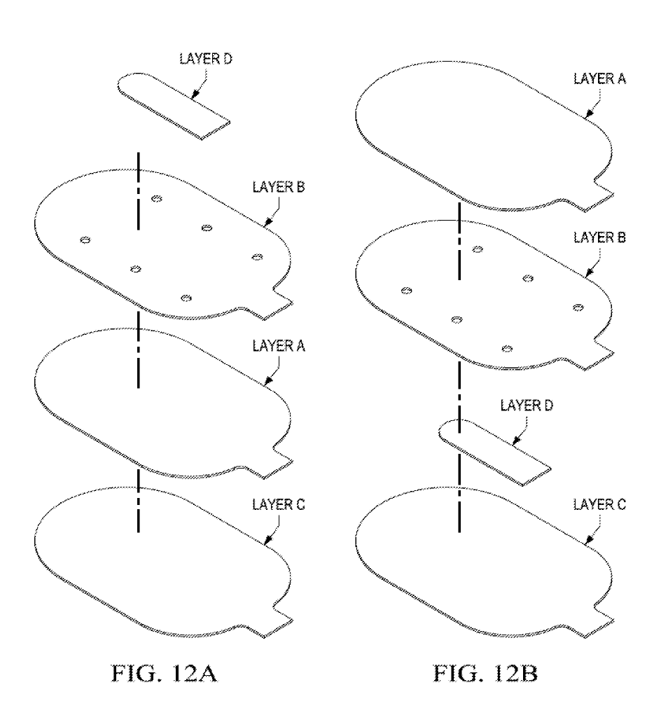

In FIG. 12A, layers B, A and C are welded (or glued or otherwise interconnected) along the perimeter. Additionally, layers D, B and A are internally welded, and preferably along the perimeter except for the open end. This way, after inversion, as shown in FIG. 12B, the space between layers D and B serves as a pocket for holding the motion sensor in close proximity to the prostate, and the unwelded open end becomes an opening to the pocket.

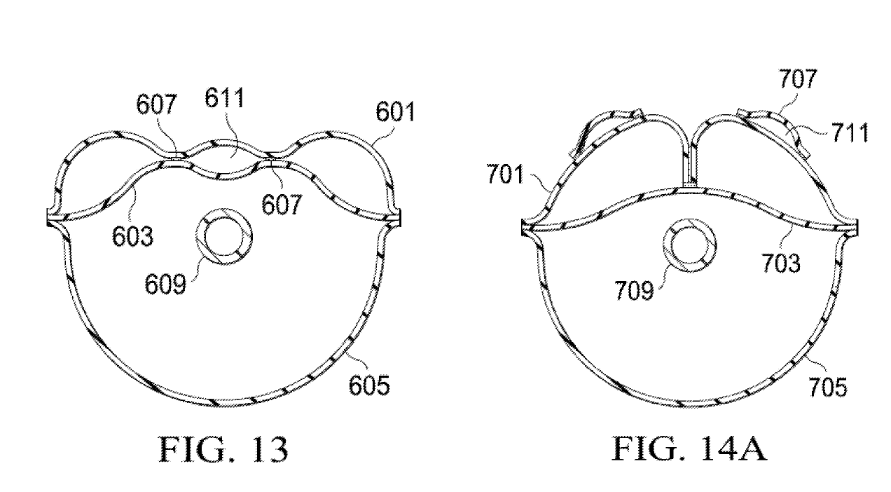

The pocket need not be made using a fourth layer, but instead the sensor can fit into the weld between the top and middle or lumen layer if that weld is U-shaped, thus leaving an opening, pocket or tunnel into which the sensor can be threaded. FIG. 13 shows such an embodiment, wherein balloon has an upper layer 601, a middle layer 603 and a lower layer 605. The layers are connected or welded at the edges to make an airtight balloon, and a lumen 609 provides an air inlet, as well as housing for the cable. The cable (not shown) exits the lumen through a hole, and then enters a pocket 611 created by the weld 607 between the upper 601 and middle 603 layers. In top view such weld would be U-shaped, the U-opening facing proximal. Although the cable is not shown in this cross section, it is similar to that shown in FIG. 11B.

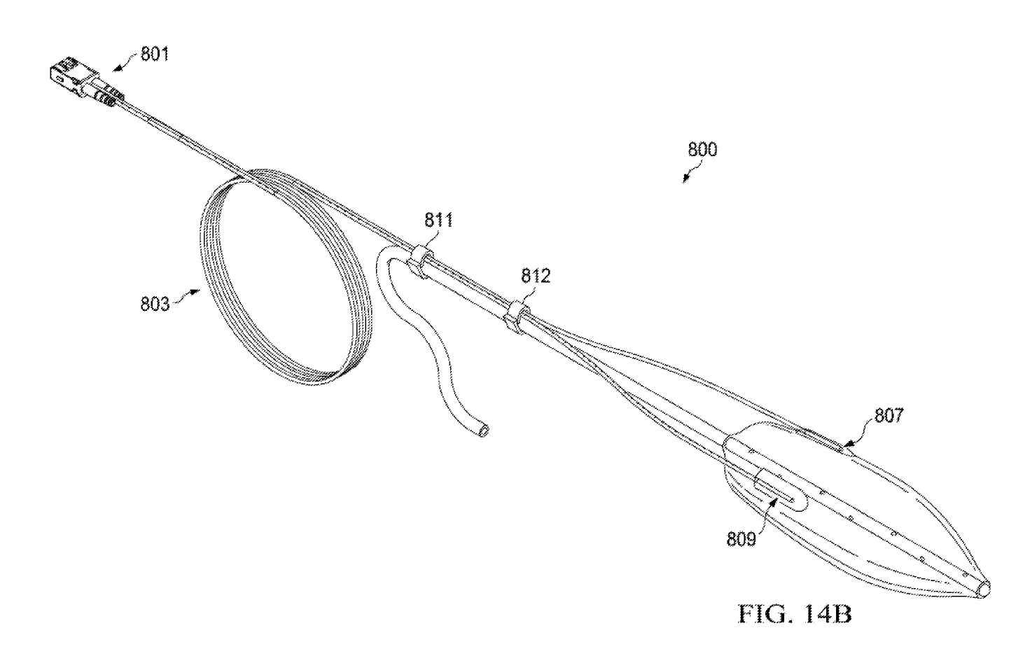

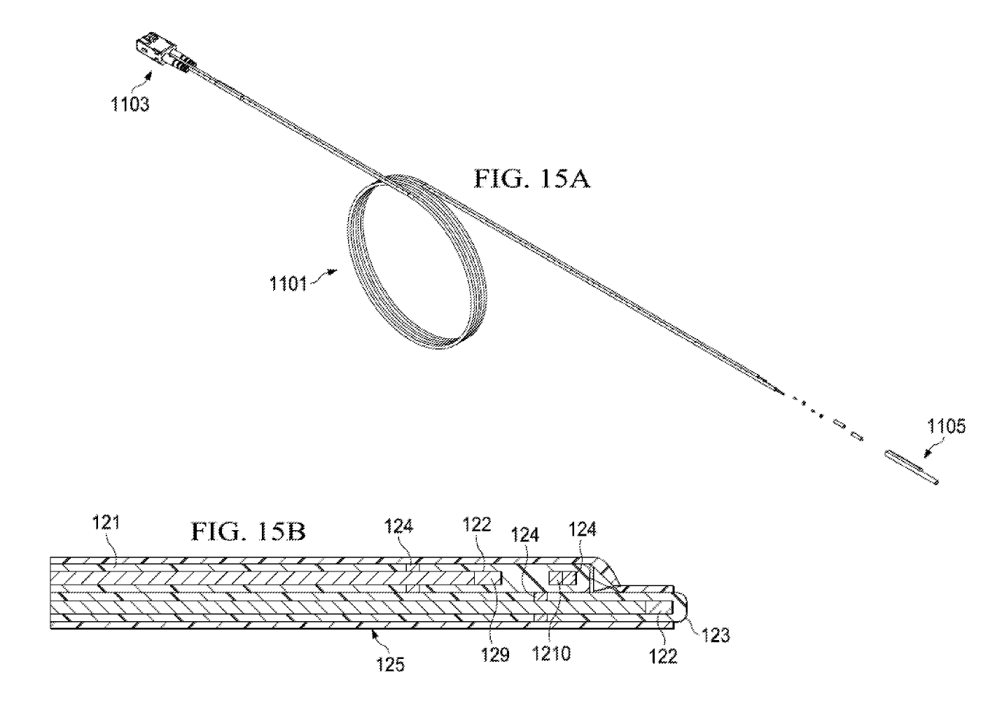

Alternatively, a pocket can be provided on the outer surface of a rectal balloon, and the pocket can lie within the dimple or groove, or a pair of pockets could pass on either side if desired. FIG. 14A shows one example of such an embodiment, wherein the upper 701, middle 703 and bottom 705 layers are edge welded, and further the upper layer 701 is attached to the middle layer 703 at a central location, thus creating a central groove or dimple into which the prostate can be cradled. A fourth layer 707 is provided for making a pocket 711 on the outer surface of the balloon. In this case there are a pair of pockets 711 on either side of the central weld, but a central pocket could be used in addition or in replacement of the pair of pockets.

FIG. 14B shows a perspective of the balloon of FIG. 14A wherein the sensor cable is bifurcates to make a pair of sensors 807 that fit into pockets 809. The cable is reversibly attached to the lumen with one or more clips 811, 812. The cable 803 is shown here coiled, and the appropriate adaptor 801 is at the proximal end for reversible and operable coupling to the reader device (not shown). In this embodiment, the cable can be detached from the balloon after use, sterilized and used again with a new balloon.

### Radiation Sensor

FIG. 15A shows the assembled radiation sensor, and FIG. 15B shows the details of the plastic scintillator, optical fiber, cap and jacket assembly.

In FIG. 15A, the radiation sensor 1101 has a SCRJ connector 1103 for connecting to a monitoring system (not shown) to read the data collected by the cable radiation sensor 1101. SCRJ connector 1103 is not detailed herein as an off the shelf part, well known to those in the art. Any suitable connector or adaptor could be used.

The radiation sensor cable 1101 also has a detecting end 1105, and the diameter of the cable should be smaller than that of the port 1009 and the tunnel 1017. When installing, the detecting end 1105 of the radiation sensor 1101 is inserted in the port 1009 into the tunnel 1017, and eventually reached the seating area 1015. The radiation sensor 1101 can further be locked in place by the hub 1011 for consistent placement. The radiation sensor cable 1101 is preferably made of flexible material due to the irregular shape of the balloon and the design of the tunnel 1017.

In more detail the detector end 1105 of the radiation sensor 1101 is shown in FIG. 15B wherein 121 is a plastic fiber optic cable, 122 is a plastic scintillating fiber being water equivalent, 123 and 129 are special caps designed to allow easier assembly of the tiny components, 124 is adhesive, 125 is heat shrinkable plastic jacket that is opaque, 1210 is a radiopaque marker bead. Additional detail can be found in US20120281945, incorporated by reference herein in its entirety.

The proximal end of the cable is outfitted with a standard coupler, in this case an SCRJ coupler, for reversible connection to a separate detector unit that detects and quantifies the signal obtained by the plastic scintillator fiber and transmitted via optic fiber to the detector unit. Any of the known detectors can be used, including a light sensor such as a photomultiplier tube (PMT), photodiode, PIN diode or CCD-based photodetector. Such device is typically connected to or outfitted with a processor and display for displaying radiation dosage to the medical practitioner.

### Motion Sensor

Motion sensors are commercially available in the art. For example, Northern Digital Inc. offers the Aurora Electromagnetic Measurement System having miniaturized sensors designed specifically for medical uses. Advantageously, no line of sight is required for this device because it does not rely on optical signals. The Aurora system (e.g., U.S. Pat. Nos. 5,923,417, 6,061,644, US20120226094, each of which is incorporated herein by reference in its entirety) includes a Field Generator (FG) that emits a low-intensity, varying electromagnetic field and establishes the position of the tracking volume. Small currents are induced in the sensors by the varying electromagnetic fields produced by the Field Generator. The characteristics of these electrical signals are dependent on the distance and angle between a sensor and the Field Generator. A Sensor Interface Units (SIU) amplifies and digitizes the electrical signals from the sensors and provides an increased distance between the System Control Unit and sensors, while minimizing the potential for data noise. The System Control Unit collects information from the SIUs, calculates the position and orientation of each sensor and interfaces with the host computer. Software is provided therewith that can be customized for the users specific applications.

In more detail, the patient is first placed within electromagnetic fields, preferably generated by the Field Generator located between the patient and the bed for treatment. The system determines the location of objects that are embedded with sensor coils. When the object (in this case a balloon having the sensor coil inside a patient) is placed inside controlled, varying magnetic fields, voltages are induced in the sensor coils. These induced voltages are used by the measurement system to calculate the position and orientation of the object, as well as being compared with prior values. As the magnetic fields are of low field strength and can safely pass through human tissue, location measurement of an object is possible without the line-of-sight constraints of an optical spatial measurement system.

One preferred sensor is the Aurora sensor 610020, which is built to order and is 2.3 mm diameter×4 mm length and can be sterilized via autoclave and is known to survive more than 20 autoclave cycles. Another preferred sensor is the Aurora sensor 610029, which is 0.8 mm diameter×9 mm length and is particularly suitable for disposable applications. Other Aurora sensors of various size and bending radius can also be used, as long as they fit within the pocket designed for the motion sensor,

In one embodiment, the motion sensor continuously monitors the location of the balloon, which serves as a surrogate method for assessing intrafraction prostate motion. The balloon allows the user (medical practitioner) to view the tip of the medical instrument, for example a flexible endoscope or in this case endorectal balloon. In this embodiment, a 6DOF sensor is provided at the tip of the apparatus, with six additional sensors distributed along the distal length. By combining this electromagnetic motion sensor with the rectal balloon apparatus, it is possible to calculate and render the apparatus' shape in real time, as well as tracking the movement of the anterior rectal wall at the rectal-prostate interface. This significantly increases the accuracy of treatment and reduces potentially serious side effect.

Further, this combination apparatus of motion sensor and rectal balloon is based on (x, y, z) navigation technology designed specifically for medical application. Based on electromagnetic technology with no line-of-sight requirements, the apparatus tracks the miniaturized sensors designed for integration into the rectal balloon device. The depth of the balloon is customized during the imaging procedure so the location of the sensor will set in a fixed location adjacent to the rectal prostatic interface.

The placement and spacing of the sensors can be customized for specific applications. In addition, the tool can be sterilized and reused, providing more economical advantages for the balloon apparatus.

The foregoing disclosure and description of the invention is illustrative and explanatory thereof Various changes in the details of the illustrated construction can be made within the scope of the present claims without departing from the true spirit of the invention. The present invention should only be limited by the following claims and their legal equivalents.

The following citations are incorporated by reference herein in their entireties for all purposes:

Flühs D, et al., Direct reading measurement of absorbed dose with plastic scintillators—the general concept and applications to ophthalmic plaque dosimetry, Med Phys. 23(3):427-304 (1996).

Beddar A S, Plastic scintillation dosimetry: optimization of light collection efficiency, Phys Med Biol. 48(9):1141-52 (2003).

Hashimoto M, Measurement of depth dose distribution using plastic scintillator, Nihon Hoshasen Gijutsu Gakkai Zasshi 59(11):1424-31 (2003).

Alcón E P, EPR study of radiation stability of organic plastic scintillator for cardiovascular brachytherapy Sr90-Y90 beta dosimetry Appl Radiat Isot. 62(2):301-6 (2/2005).

Tanderupa K., et al. In vivo dosimetry in brachytherapy, Med. Phys. 40 (7) (2013).

Mijnheer, B. et al., In vivo dosimetry in external beam radiotherapy, Med. Phys. 40 (7) (2013).

U.S. Pat. No. 8,080,031 Minimally invasive rectal balloon apparatus and related cases U.S. Pat. Nos. 8,454,648, 8,500,771, 8,241,317, US20120123185, US2012078177, US20120259197

US20130123621 Dual chamber irradiation balloons

US20120281945 Small diameter radiation sensor cable

US20130109906 Shaped Conforming Medical Balloons

US20130085315 Universal balloon for brachytherapy applicator

US20030028097 Immobilizer probe system and method

U.S. Pat. No. 6,963,771 Methods, systems, and associated implantable devices for radiation dose verification for therapies used to treat tumors

U.S. Pat. No. 7,361,134 Method and apparatus for real time dosimetry

U.S. Pat. Nos. 7,662,083 and 8,133,167 Apparatus and method for brachytherapy radiation distribution mapping

US20120068075 Real-time in vivo radiation dosimetry using scintillation detectors

U.S. Pat. No 8,183,534 Scintillating fiber dosimeter array

U.S. Pat. No. 5,923,417 System for determining the spatial position of a target

US6061644 System for determining the spatial position and orientation of a body

US20120226094 Targeting orthopedic device landmarks

WO2013024380 Method to estimate interfractional and intrafractional organ motion for adaptive external beam radiotherapy

1. A device comprising:
a shaft comprising a shaft proximal end, a shaft distal end, an inflation lumen and a gas release lumen;
a balloon comprising a balloon proximal end and a balloon distal most end;
the inflation lumen configured to be in fluid communication with the balloon;
the gas release lumen comprising a gas release lumen proximal end and a gas release lumen distal end, wherein the gas release lumen distal end extends a selected distance distally beyond the balloon distal most end.

2. The device of claim 1, further comprising at least one pocket on an outer surface of the balloon.

3. The device of claim 2, wherein the at least one pocket is configured to receive at least a section of at least one sensor.

4. The device of claim 3, wherein the at least one sensor is a motion sensor.

5. The device of claim 3, wherein the at least one sensor is a radiation sensor capable of determining the amount of radiation delivered during a radiation treatment.

6. The device of claim 1, wherein the shaft further comprises at least one sidehole providing for fluid communication between the gas lumen and the atmosphere.

7. The device of claim 6, wherein the at least one sidehole is positioned a selected distance distally beyond the distal most end of the balloon.

8. The device of claim 2, further comprising at least two pockets on a surface of the balloon.

9. The device of claim 5, wherein the radiation sensor further comprises a plastic scintillator fiber coupled to an optical cable.

10. The device of claim 3, wherein the at least one sensor extends coaxially within a tunnel of the shaft.

11. A device comprising:
a shaft comprising a shaft proximal end, a shaft distal end, a shaft length extending between the shaft proximal end and the shaft distal end, an inflation lumen and a gas release lumen;
a unitary balloon comprising a unitary balloon proximal end and a unitary balloon distal most end, wherein both the unitary balloon proximal end and the unitary balloon distal most end are secured to the shaft; and
the shaft further comprising an inflation lumen distal most end, a sensor tunnel, and a gas release lumen distal most end, wherein the inflation lumen distal most end terminates a selected distance proximal to the balloon distal most end and the gas release lumen distal most end terminates a selected distance distally beyond the balloon distal most end.

12. The device of claim 11, wherein the shaft further comprises a shaft longitudinal axis, wherein the sensor tunnel further comprises a rise portion, wherein the rise portion extends away from the shaft longitudinal axis for a selected distance.

13. 1 The device of claim 12, further comprising at least two sensors, wherein the two sensors extend coaxially along the shaft longitudinal axis for a selected distance.

14. 1 The device of claim 13, wherein the at least two sensors comprise a plastic scintillator fiber coupled to an optical cable.

15. The device of claim 14, wherein one of the at least two sensors comprise a motion sensor.

16. The device of claim 14, wherein one of the at least two sensors is a radiation sensor capable of determining the amount of radiation delivered during a radiation treatment.

17. The device of claim 16, wherein the sensor is capable of determining the amount of radiation delivered during a radiation treatment.

18. A device comprising:
a shaft comprising a shaft proximal end, a shaft distal end, an inflation lumen, a shaft longitudinal axis, and a sensor tunnel, wherein the sensor tunnel further comprises a rise portion, wherein the rise portion extends away from the shaft longitudinal axis for a selected distance;
a balloon comprising a balloon proximal end and a balloon distal most end, wherein the rise portion extends within an interior of the balloon;
the inflation lumen configured to be in fluid communication with the balloon;
a first sensor extending coaxially along the sensor tunnel.

19. The device of claim 18, further comprising a fiducial marker located on the shaft distal end, the first sensor comprising a first sensor distal end, wherein the first sensor distal end is located a selected distance proximal to the fiducial marker.

20. The device of claim 18, further comprising a second sensor extending coaxially along the shaft longitudinal axis.
